(19) United States
(12) Patent Application Publication
(10) Pub. No.: US 2013/0144325 A1
(43) Pub. Date: Jun. 6, 2013

(54) NASAL DILATOR

(71) Applicant: Ludwig A. Allegra, Sammamish, WA (US)

(72) Inventor: Ludwig A. Allegra, Sammamish, WA (US)

(21) Appl. No.: 13/487,359

(22) Filed: Jun. 4, 2012

Related U.S. Application Data
(60) Provisional application No. 61/492,687, filed on Jun. 2, 2011.

(51) Int. Cl. A61F 5/08

(52) U.S. Cl. CPC A61F 5/08
USPC 606/199

(57) ABSTRACT

Described are magnets, dilating members comprising magnets, and nasal dilators for nasal dilatation that dynamically maintain fit and dilatation in a user's nostrils without manual intervention. Also described are kits comprising multiple pairs of magnets for assembling a nasal dilator to meet a user's requirements.

### CROSS-REFERENCE TO RELATED APPLICATION

This application claims priority to and the benefit of U.S. Provisional Application Ser. No. 61/492,687, filed Jun. 2, 2011, entitled “Nasal Dilator,” the disclosure of which is hereby incorporated by reference herein in its entirety.

### TECHNICAL FIELD

The present invention generally relates to nasal dilators comprising magnets that maintain fit and function in a user's nostrils without manual adjustment.

### BACKGROUND

A significant portion of the population experiences partial or complete nasal congestion, blockage, and/or obstruction (generally, nasal constriction) at some point in their lifetime. The causes of nasal constriction can range from mild conditions such as allergic reactions, common cold, sinusitis, and deviated septum, to more severe conditions such as nasal polyps and reaction to medication. Similarly, the effects of nasal constriction can range from mild annoyance, snoring, headaches and/or facial pain, to sleep apnea, and life-threatening conditions such as hypoxia, right-sided heart failure, and respiratory distress (in infants, for example).

One method of treating nasal constriction is with the use of nasal dilators, which work by opening the nostril or nasal passage to improve airflow through the nose. Nasal dilators may be broadly classified as external (e.g., by attachment to the skin of the nose) or insertable (by insertion and engagement with the inner lining(s) of the nostril). Common problems associated with both types of nasal dilators include: lack of flexibility for a wide variety of noses and sizes, irritability of the nose and/or the nostril lining, lack of reusability, and discomfort. It would therefore be desirable to provide new and improved nasal dilator and methods that overcome some or all of these and other drawbacks.

### SUMMARY OF THE INVENTION

Devices, methods and kits for dilation of a user's nostrils are disclosed herein. In one embodiment, a nasal dilator includes a first dilating member configured to be disposed in a first nostril of a user, a second dilating member configured to be disposed in a second nostril of the user, and a support structure coupled to the first dilating member and the second dilating member. The first and second dilating member can be at least partially formed of a magnet or can be configured to receive a removable magnet and the support structure is configured to position the first dilating member relative to the second dilating member such that when the dilating members are disposed in the nostrils of the user, the repulsive force between the magnets holds the first dilating member in pressure contact with a first nostril and second dilating member in pressure contact with a second nostril.

### BRIEF DESCRIPTION OF THE DRAWINGS

FIG. 1A is a schematic illustration of a nasal dilator according to an embodiments.

FIG. 1B is a top view of the nasal dilator of FIG. 1.

FIG. 2A is a perspective view of a nasal dilator according to an embodiment.

FIG. 2B is cross-sectional view taken along the plane 2B-2B of FIG. 2A.

FIG. 2C is an illustration of the nasal dilator of FIG. 2A inserted into the nostrils of a patient.

FIGS. 3-7 are perspective views of nasal dilators according to additional embodiments.

### DETAILED DESCRIPTION

Devices, methods and kits for dilation of a user's nostrils are disclosed herein. In one embodiment, a nasal dilator includes a first dilating member including a first magnet configured to be disposed in a first nostril of a user, and a second dilating member including a second magnet configured to be disposed in a second nostril of the user. The first and the second dilating members and/or magnets can be contoured or otherwise shaped to fit into the nostrils of a patient. Said another way, the dilating members can come in different sizes, shapes and/or be customizable to improve comfort for the patient. The nasal dilator also includes a support structure coupled to the first dilating member and the second dilating member configured to position the first dilating member relative to the second dilating member such that when disposed in the nostrils of the user, the repulsive force between the first and second magnets holds the first dilating member in pressure contact with the first nostril and second dilating member in pressure contact with the second nostril. The repulsive force between the two magnets opens the nasal passages to improve airflow through the nose and the frictional engagement between the dilating members and nostrils holds the nasal dilator in position.

In some embodiments, the first and second magnets are permanent magnets such as, for example, rare-earth magnets. Rare-earth magnets including neodymium-iron-boron and samarium-cobalt magnets produce a significantly stronger magnetic field than other types of magnets such as, for example, ferrite or alnico magnets. The remanence, or strength of the magnetic field, of rare-earth magnets can be in excess of 1.4 teslas, whereas ferrite or ceramic magnets typically exhibit fields in the range of 0.5 to 1 tesla. In some embodiments, the first and second magnets independently have a remanence of at least about 0.6 tesla, of at least about 0.7 tesla, of at least about 0.8 tesla, of at least about 0.9 tesla, of at least about 1.0 tesla, of at least about 1.1 tesla, of at least about 1.2 tesla, of at least about 1.3 tesla, of at least about 1.4 tesla, and all values in-between.

The term “about”, as used in this description to describe a numerical value, and unless stated otherwise, refers to a range about and including the described numerical value, wherein the range is determined by one or more of: experimental error in measuring the numerical value, and ±10% of the numerical value.

Rare-earth magnets typically also have higher energy per unit volume (also termed as “energy density” or “energy product”) than other types of magnets. In other words, a smaller size or volume of a rare earth magnet can be employed compared to other magnets to achieve a target energy product. In some embodiments, the first and second magnets independently have an energy product of at least about 60 kJ/m³, at least about 70 kJ/m³, at least about 80 kJ/m³, at least about 90 kJ/m³, at least about 100 kJ/m³, at least about 150 kJ/m³, at least about 200 kJ/m³, at least about 250 kJ/m³, at least about 300 kJ/m³, at least about 350 kJ/m³, at least about 400 kJ/m³, at least about 450 kJ/m³, at least about 500 kJ/m³, and all values in-between.

Rare earth magnets also maintain their magnetization better in the presence of a demagnetizing force or field, and hence are beneficial for long term use without undue concern about other magnetic fields that might be encountered by the user. In some embodiments, the coercivity (resistance to demagnetization) of the first and second magnets is independently at least about 300 kiloamperes/meter (kA/m), at least about 350 kA/m, at least about 400 kA/m, at least about 450 kA/m, at least about 500 kA/m, at least about 550 kA/m, at least about 600 kA/m, at least about 700 kA/m, at least about 800 kA/m, at least about 900 kA/m, at least about 1000 kA/m, at least about 1200 kA/m, at least about 1400 kA/m, at least about 1600 kA/m, at least about 1800 kA/m, at least about 2000 kA/m, and all values in-between.

In some embodiments, the rare earth magnets may be produced via a sintering process, or via a bonding process, as is known in the art. In some embodiments, the rare earth magnets are produced via a sintering process, and may include a protective coating to prevent corrosion. Any suitable protective coating may be using, including metal compositions (e.g. gold, copper, or nickel), lacquer, and/or a compliant polymer.

An example of rare-earth magnets that can be used in the nasal dilator described herein include neodynium magnets, such as ¼″× 1/16″ Disc Rare Earth Neodymium Magnets obtained from Applied Magnets® that are composed of neodynium, iron, and boron, have a nickel-copper-nickel triple layer coating, and have a pull force of 1.6 lbs.

In some embodiments, each dilating member is configured to improve retention of the nasal dilator in the patient's nostrils. Said another way, the dilating members can be formed of a material, coated, covered, or otherwise treated to increase the frictional coefficient between the surface or surfaces contacting the nostrils, and the nostrils. The dilating members may be textured, by hatching the surfaces of the first and second magnet for example. In another example, the dilating members can be coated with a slip resistant material, such as a viscous gel. In some embodiments, the frictional coefficient can be increased by the use of a compliant covering. The compliant covering deforms upon pressure contact with the nostrils, which increases the surface area of interaction between the compliant covering and the nostril, which in turn increases friction. In some embodiments, the compliant covering can be removable to allow periodic replacement of the coverings, for purposes of effectiveness and/or hygiene, for example. An additional or alternative approach to increasing friction is the use of adhesive materials, and/or coverings.

In some embodiments, a suitable chemical may be incorporated into the compliant covering for intranasal delivery. For example, if the patient is suffering from a respiratory bacterial infection which is causing the nasal constriction or obstruction, an antimicrobial agent might be employed. In another example, if the patient is experiencing facial pain, delivery of a painkiller via the coating and/or covering on the dilating members might function as a local anesthetic to alleviate the pain. In some embodiments, the coatings and/or covering can include counterirritants such as, for example, menthol to facilitate direct, more efficient delivery of the counterirritant directly into the nostrils, rather than the more common approaches of oral tablets and inhalation.

In some embodiments, a nasal dilator includes a first dilating member having a recess configured to receive a removable first magnet, and a second dilating member having a recess configured to receive a removable second magnet. The nasal dilator also includes a support structure coupled to the first dilating member and the second dilating member. The support structure has a first configuration when the first and second magnets are disposed in their respective recesses and a second configuration when at least one of the first and second magnets is removed from its respective recesses. The support structure positions the first and second dilating members such that when both magnets are disposed in their respective recesses, the support structure is in a first configuration due to the repulsive force of the magnets. Said another way, the repulsive force of the two magnets causes the two dilating members to separate and expand the support structure. When one of both magnets is removed from their respective recesses, the support structure is in a second configuration where there is no magnetic repulsive force between the dilating members. In the second configuration, due to the lack of repulsive forces, the support structure can collapse or be collapsed. When both magnets are disposed in their respective recesses and the dilator is placed in the user's nose, the support structure is in a third configuration whereby the first and second dilating members are in pressure contact with the user's nostrils. Said another way, the third configuration can be characterized as a partially expanded configuration such that there is sufficient repulsive force to frictionally engage the user's nostrils and open the nasal passages to improve airflow through the user's nose.

In some embodiments, each dilating member can include a compliant material that is configured to improve retention of the nasal dilator in the patient's nostrils. For example, a recess forming portion of the dilating members can be formed of a material that increases friction between the recess forming portion and the nostrils. In some embodiments, friction is increased by the use of a compliant material for making the recess forming portion. The compliant material can deform upon pressure contact with the nostrils, which in turn increases friction. In some embodiments, friction is increased by using compliant materials that are also adhesive in nature. In some embodiments, the compliant material is removable for periodic replacement. In other words, the recess forming portion of the dilating member can be disengaged and replaced.

In some embodiments, the nasal dilators described herein can be included in a kit. For example, to account for variations in factors such as nose size, tolerance to pressure generated by the nasal dilator, severity of the nasal obstruction, etc., a kit can include a nasal dilator configured for receiving removable magnets and a plurality of pairs of removable magnets, each pair having a different magnetic strength. In some embodiments, the kit can include more than one nasal dilator, each nasal dilator of a different size to accommodate different nose sizes. In some embodiments, the kit can include several nasal dilators of the same size, each having a pair of magnets coupled thereto as described above, of different strength. The kit of several nasal dilators described herein may further include multiple compliant coverings of different sizes. Indeed, any combination of one or more removable/attached magnets, one or more nasal dilators, and one or more compliant coverings may be employed to accomplish the purpose of fit and effectiveness. In some embodiments, a code is used to distinguish between pairs of magnets of different strengths, between dilators of different sizes, between dilators of different strength (i.e. having magnets coupled thereto), and/or between compliant coverings of different sizes. In some embodiments, the code is a color code. In some embodiments, the code is alphanumeric.

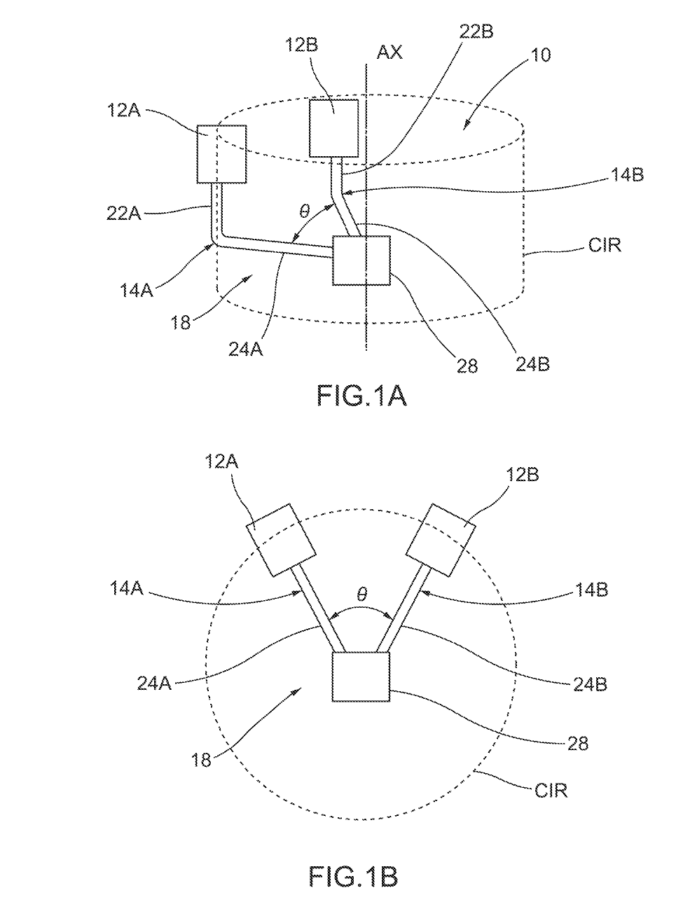

An insertable nasal dilator 10 (also referred to herein as “dilator”) according to an embodiment is schematically illustrated in FIGS. 1A and 1B. The dilator 10 includes a first dilating member 12A, a second dilating member 12B, and a support structure 18 coupled to the first dilating member and the second dilating member. The first dilating member 12A is configured to be disposed in a first nostril of a user, while the second dilating member 12B is configured to be disposed in a second nostril of the user. During use, the dilator 10 exerts a pressure for providing gentle dilatation of the nostrils, while also holding the dilator in pressure contact with the nostrils to prevent fallout or loosening such as, for example, during flaring of the nostrils.

In some embodiments, the first dilating member 12A includes a first magnet, and the second dilating member 12B includes a second magnet. Each magnet has a magnetic polarity, i.e., comprises a north magnetic pole (“north pole”) and a south magnetic pole (“south pole”). The concept of magnetic poles of magnets and that of attraction/repulsion between unlike/like poles is well known, and thus not described in detail herein. In some embodiments, the support structure 18 is configured to position the first dilating member 12A relative to the second dilating member 12B such that when the dilating members 12A and 12B are disposed in the nostrils of the user, a repulsive force exists between the first and second magnets. Said another way, the first and the second magnets are positioned by the support structure 18 such that the same poles (e.g., north-north or south-south) are adjacent causing the magnets to repel each other. The repulsive force tends to increase the separation between the first and second magnets, thereby pressing the first and second dilating members against the first and second nostrils, respectively. This pressing action opens the nasal passages to improve airflow through the nose and holds the first dilating member 12A in pressure contact with the first nostril and second dilating member 12B in pressure contact with the second nostril.

In some embodiments, the support structure 18 comprises a first arm 14A coupled to the first dilating member 12A and a second arm 14B coupled to the second dilating member 12B. In some embodiments, the dilating members 12A, 12B are detachable from (i.e. may be removably attached to) the arms 14A, 14B. In some embodiments, each arm 14A, 14B includes a first portion 22A, 22B configured to be attached to the dilating members 12A, 12B and further configured to be disposed in the respective nostril. In this manner, when the first dilating member 12A is disposed in the first nostril of the user and the second dilating member 12B is disposed in the second nostril of the user, repulsion between the first portions 22A, 22B holds the first dilating member 12A in pressure contact with the first nostril and the second dilating member 12B in pressure contact with the second nostril, thereby holding the nasal dilator 10 in place. In other words, it is the intranasal components of the nasal dilator 10 that perform the dual function of dilatation and maintaining fit.

In some embodiments, each arm 14A, 14B includes a second portion 24A, 24B configured to be disposed outside the respective nostril. In some embodiments, the arms 14A, B are pliable and/or ductile, and allow for independent and manual adjustment of the relative lengths and/or thickness of the first portions 22A, 22B and the second portions 24A, 22B. In this manner, a user may adjust the depth of penetration of the dilating members 12A, 12B into the user's nostrils. In some embodiments, the material of the arms 14A, 14B is biocompatible. Any suitable material that imparts pliability, ductility, and/or biocompatibility may be employed to construct the arms 14A, 14B including, but not limited to, metals, plastics, composites, and combinations thereof.

In some embodiments, the support structure 18 further comprises a connector 28 for attaching, joining, interconnecting, linking, or otherwise combining the first arm 14A and the second arm 14B in any suitable manner. Any design of the connector 28 that permits, in response to the repulsion between the dilating members 12A, 12B as described herein, bidirectional motion of each arm 14A, 14B is within the scope of the invention.

In some embodiments, the bidirectional motion of the arms 14A, 14B is lateral. In some embodiments, the bidirectional motion of the arms 14A, 14B is rotary. For example, as illustrated in FIGS. 1A-B, the arms 14A, 14B may form an arm angle ⊖ at the connector 28 which increases when repulsion between dilating members 12A, 12B leads to relative rotary motion between the arms. A maximum arm angle is achieved when no further increase in separation between the arms 14A, 14B is possible such as, for example, due to design characteristics of the connector 28 or a limit on the repulsive force of the magnets. In some embodiments, the maximum arm angle is about 180°. In some embodiments, the maximum arm angle is about 170°, is about 160°, is about 150°, is about 140°, is about 130°, is about 120°, is about 110°, is about 100°, is about 90°, is about 70°, is about 60°, is about 50°, is about 40°, is about 30°, and all values in-between. In some embodiments, the arm angle is in the range of about 30° to about 90°. The dilator 10 can be considered to be in a stable or resting configuration when the arm angle ⊖ is about the same as the maximum arm angle. Prior to use, the user may deform the dilator 10 by forcing the arms 14A, 14B closer together for insertion of the dilation members 12A, 12B into the user's nostrils. In this state, the dilator 10 may be considered to be in a contracted, or partially contracted configuration, where the arm angle ⊖ is less than the maximum arm angle. A plurality of contracted configurations are achievable, corresponding to different values of the arm angle ⊖. In some embodiments, the dilator 10 can only have discrete, discontinuous values of the arm angle ⊖ during deformation, and the plurality of contracted configurations are hence discrete. In some embodiments, continuous variation of the arm angle ⊖ is possible, and the plurality of contracted configurations are hence continuous.

In some embodiments, the arms 14A, 14B, and particularly the first portions 22A, 22B, move in a plane CIR with a center axis AX. In some embodiments, the center axis AX is defined by the connector 28. In some embodiments, the plane CIR is cylindrical. In some embodiments, the plane CIR is a conical frustum. In some embodiments, the plane CIR is an inverted conical frustum. The arms 14A, 14B can maintain their alignment in the plane CIR while moving away from one another (i.e. independent of the arm angle ⊖), thereby allowing lateral dilatation over the entire range of operation up to the maximum arm angle, while maintaining rotational stability.

In some embodiments, the connector 28 is formed by combining the arms 14A, 14B alone. In some embodiments, the connector 28 is an additional structure or component. The attachment of the connector 28 to each arm 14A, B can be independent of the other arm. In some embodiments, the connector 28 can be one or more of the following: an intertwined structure formed of the arms 14A, B, a connecting loop, a sleeve configured to receive at least a portion of the arms 14A, 14B, a separate sleeve for receiving at least a portion of each arm, one or more annular rings, and a hinge mechanism. In some embodiments, the arms 14A, 14B are detachable from the connector 28. In. some embodiments, the connector 28 is integrally formed with the arms 14A, 14B.

In some embodiments, one or more components of the dilator 10 (i.e. the dilating members 12A-B, the arms 14-B, and/or the connector 28) can be ornamental/decorative, and/or include ornamental/decorative components. In some embodiments, the ornamental/decorative components are precious or semi-precious elements, including precious stones, platinum, gold, silver, and combinations thereof.

In some embodiments, the dilator 10 can be included in a kit comprising a plurality of pairs of insertable magnets, a first dilating member 12A and a second dilating member 12B, each configured for removable insertion of one magnet of a pair of the insertable magnets, and a support structure 18. In some embodiments, each insertable magnet comprises at least one or more rare earth metal. In some embodiments, each pair of magnets has the same color code. In some embodiments, each pair of magnets has a different color code than each other pair of magnets. The magnet color codes may be used to distinguish between separate pairs of magnets based on any suitable characteristic that would affect a user's comfort, fit, dilatation needs, and so on. Examples of such suitable characteristics include, but are not limited to, one or more of the following: length, width, thickness, curvature, magnetic strength, biocompatible coating, and combinations thereof. In some embodiments, the support structure 18 includes arms 14A, 14B and a connector 28, as described herein.

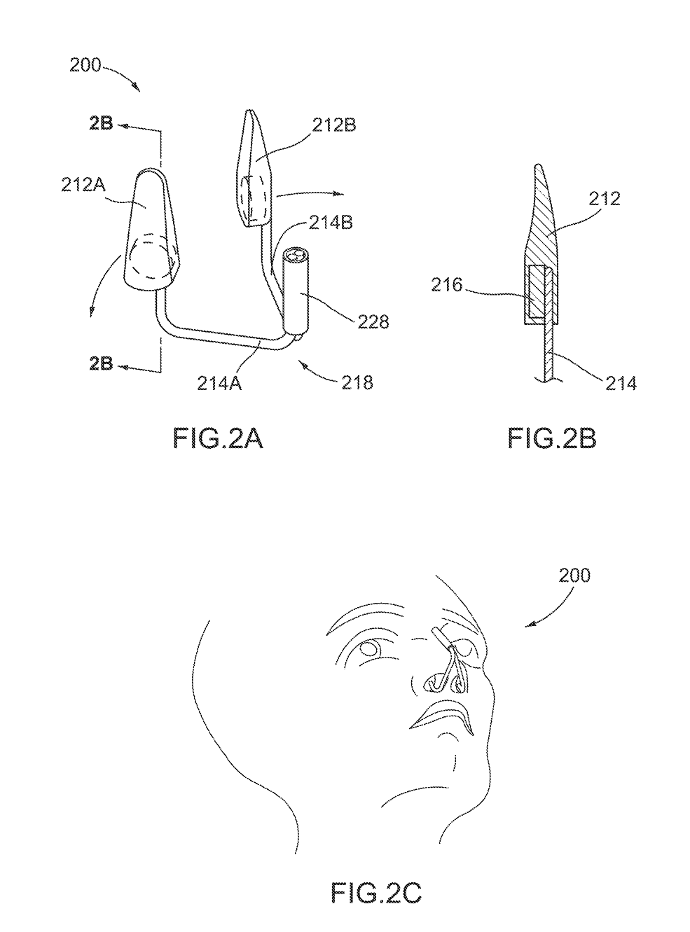

FIGS. 2A-7 illustrate various support structures and connectors according to embodiments. Unless stated otherwise, it is understood that various components illustrated in these figures correspond substantially to similarly named and referenced components in FIG. 1. For example, arms 214A and 214B in FIGS. 2A and 2B are similar to arms 14A and 14B, and so on.

FIG. 2A illustrates a dilator 200 where a connector 228 is formed as a sleeve for arms 214A-B. FIG. 2B illustrates an arm 214 (corresponding to any of the arms 214A, B) designed as a biocompatible holder with a slot 216 for holding a magnet. FIG. 2C illustrates the dilator 200 during use.

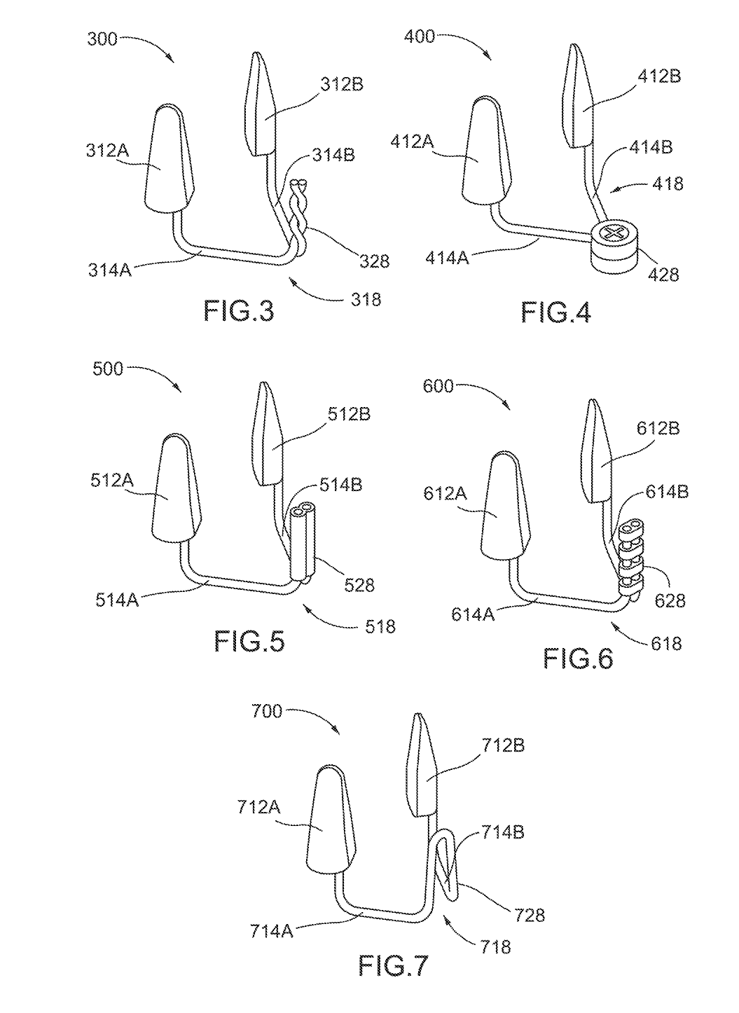

The embodiments of FIGS. 3-7 illustrate different designs of the connector 18 of FIG. 1. FIG. 3 illustrates the connector as an intertwining structure 318 formed by the arms 314A, 314B. FIG. 4 illustrates the connector formed as a barrel hinge 418. FIG. 5 illustrates the connector formed as a sleeve 518 with separate conduits for each arm 514A, 514B. FIG. 6 illustrates the connector formed as a plurality of sleeves 618, each with a separate conduit for the arms 614A, 614B. The plurality of sleeves 618 may be interconnected (not shown). FIG. 7 illustrates an embodiment where the arms 714A, 714B are integrally formed with each other and where the connector is an interconnecting loop 718 to facilitate ease of manipulation by the user.

Aspects of the invention hence provide for rare-earth element based magnets and dilating members for continual, lateral nasal dilatation while dynamically maintaining fit, without manual intervention. The use of rare-earth magnets, which are typically of higher magnetic strength compared to other magnetic materials, provides effective repulsive forces without the use of large magnets within the nostril, which would otherwise restrict airflow. Aspects of the invention also provide for kits having multiple pairs of magnets, which permit a user to assemble a dilator most suitable to his/her needs.

The various embodiments described herein should not to be construed as limiting this disclosure in scope or spirit. It is to be understood that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

For example, and referring again to FIG. 1 but with relevance to any of the above-mentioned embodiments, it is possible that the dilating members 12A, 12B be formed along the length of the arms 14A, 14B, instead of at the end as illustrated. In such embodiments, the dilating members 12A, 12B would still be operable as described above. In some embodiments, the dilating members 12A, 12B may be formed on the second portions 24A, 24B. In other words, the dilating members 12A, 12B would be disposed outside the nostril during use. In this manner, the footprint of the dilator 10 inside the nostril may be significantly reduced. In some embodiments where the dilating members 12A, 12B are formed on the portions 24A, 24B, the dilating members may be removable. In some embodiments, a pair of lateral arms (not shown), one on each second portion 24A, 24B, is present to attach or have formed thereon the dilating members 12A, 12B in any suitable manner. In some embodiments, the user may remove and/or replace the dilating members 12A, 12B without removing the dilator 10, and/or by manually holding the dilator in place while replacing the dilating members.

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A nasal dilator, comprising:
a first dilating member configured to be disposed in a first nostril of a user, the first dilating member including a first magnet;
a second dilating member configured to be disposed in a second nostril of the user, the second dilating member including a second magnet; and
a support structure coupled to the first dilating member and the second dilating member, the support structured configured to position the first dilating member relative to the second dilating member such that when disposed in the nostrils of the user, the repulsive force between the first and second magnets holds the first dilating member in pressure contact with the first nostril and second dilating member in pressure contact with the second nostril.

2. The nasal dilator of claim 1, wherein the first magnet and the second magnet are rare-earth magnets.

3. The nasal dilator of claim 2, wherein the first and the second magnets each have a coercivity of at least about 60 kJ/m³.

4. The nasal dilator of claim 2, wherein the first and the second magnets each have a coercivity of at least about 200 kJ/m³.

5. The nasal dilator of claim 2, wherein the first and the second magnets each have a remanence of at least about 0.6 tesla.

6. The nasal dilator of claim 2, wherein the first and the second magnets each have a remanence of at least about 1 tesla.

7. The nasal dilator of claim 2, wherein the first and second magnets each have a protective coating formed thereon to prevent corrosion.

8. The nasal dilator of claim 1, further comprising a compliant covering disposed on each of the first dilating member and the second dilating member, the coverings configured to conform to the contours of the nostrils of the user.

9. The nasal dilator of claim 8, wherein the compliant covering is removable.

10. The nasal dilator of claim 8, wherein the compliant covering has antimicrobial properties.

11. The nasal dilator of claim 8, wherein the compliant covering has a surface treatment configured to enhance frictional engagement with the nostrils of the user.

12. A nasal dilator, comprising:
a first dilating member configured to be disposed in a first nostril of a user, the first dilating member having a recess configured to receive a removable first magnet;
a second dilating member configured to be disposed in a second nostril of the user, the second dilating member having a recess configured to receive a removable second magnet; and
a support structure coupled to the first dilating member and the second dilating member, the support structure having a first configuration when the first and second magnets are disposed in their respective recesses, and a second configuration when at least one of the first and second magnets is removed from its respective recess.

13. The nasal dilator of claim 12, wherein the first and second dilating members include a compliant material configured to conform to the contours of the user's nostrils.

14. The nasal dilator of claim 12, wherein the support structure includes a first arm coupled to the first dilating member, a second arm coupled to the second dilating member, and a connector coupled to the first arm and the second arm.

15. The nasal dilator of claim 14, wherein the connector is configured to allow bidirectional motion of the first and the second arm.

16. The nasal dilator of claim 14, wherein the first arm and the second arm are rotatably coupled to the connector.

17. The nasal dilator of claim 16, wherein the first arm and the second arm define an arm angle, the support structure having a maximum arm angle in a first configuration.

18. The nasal dilator of claim 14, wherein the first arm and the second arm are detachable from the connector.

19. The nasal dilator of claim 14, wherein the connector includes at least one of an intertwined structure formed of the first arm and the second arm, a connecting loop, a sleeve configured to receive at least a portion of the first arm and the second arm, a plurality of annular rings, and a hinge mechanism.

20. The nasal dilator of claim 14, wherein each arm is pliable to vary the relative length of a first portion of the each arm disposed within the nostril of the user and a second portion of the each arm disposed outside the nostril of the user.

21. A kit, comprising:
a nasal dilator including:
a first dilating member configured to be disposed in a first nostril of a user, the first dilating member configured to receive a first magnet of a selected pair of magnets;
a second dilating member configured to be disposed in a second nostril of the user, the second dilating member configured to receive a second magnet of the selected pair of magnets; and
a support structure coupled to the first dilating member and the second dilating member, the support structure configured to position the first dilating member relative to the second dilating member when disposed in the nostrils of the user; and
a plurality of pairs of insertable magnets.

22. The kit of claim 21, wherein each of the plurality of pairs of insertable magnets are rare-earth magnets.

23. The kit of claim 22, wherein each pair of the plurality of insertable magnets has a different repulsive force.

24. The kit of claim 23, wherein the plurality of pairs of insertable magnets are color coded according to their respective repulsive force.
